(19) United States
(12) Patent Application Publication
(10) Pub. No.: US 2005/0276862 A1
(43) Pub. Date: Dec. 15, 2005

(54) Iron sequestering antimicrobial composition

(76) Inventors: Joseph F. Bringley, Rochester, NY (US); Yannick J. Lerat, Mellecey (FR)

Correspondence Address: Paul A. Leipold;Patent Legal Staff, Eastman Kodak Company, 343 State Street, Rochester, NY, 14650-2201, US

(21) Appl. No.: 10/868,626

(22) Filed: Jun. 15, 2004

(51) Int. Cl. A61K 9/16

(52) U.S. Cl. 424/490

(57) ABSTRACT

This invention relates to a composition of matter comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion. It further relates to articles comprising said composition and to a method of removing target metal-ions from an environment and releasing antimicrobial metal ions into the environment comprising contacting the environment with said composition

### CROSS REFERENCE TO RELATED APPLICATIONS

This application relates to commonly assigned, concurrently filed, co-pending application Ser. No. ______ (Docket No. 86559A) entitled “Antimicrobial Metal-Ion Sequestering Web for Application to a Surface” which is a CIP of Ser. No. 10/737,346 entitled “An Iron Sequestering Antimicrobial Composition” which was filed Dec. 16, 2003. These co-pending applications are incorporated by reference herein for all that they contain.

### FIELD OF THE INVENTION

This invention relates to a composition comprising a metal ion sequestrant and an antimicrobial metal ion. It further relates to an article comprising the composition and a method of removing metal contaminants from a surrounding environment.

### BACKGROUND OF THE INVENTION

In recent years people have become very concerned about exposure to the hazards of microbe contamination. For example, exposure to certain strains of Escherichia coli through the ingestion of under-cooked beef can have fatal consequences. Exposure to Salmonella enteritidis through contact with unwashed poultry can cause severe nausea. Mold and yeast (Candida albicans) may cause skin infections. In some instances, biocontamination alters the taste of the food or drink or makes the food unappetizing. With the increased concern by consumers, manufacturers have started to produce products having antimicrobial properties. A wide variety of antimicrobial materials have been developed which are able to slow or even stop microbial growth; such materials when applied to consumer items may decrease the risk of infection by microorganisms.

Noble metal-ions such as silver, copper and gold ions are known for their antimicrobial properties and have been used in medical care for many years to prevent and treat infection. In recent years, this technology has been applied to consumer products to prevent the transmetal-ion sequestrantion of infectious disease and to kill harmful bacteria such as Staphylococcus aureus and Salmonella. In common practice, noble metals, metal-ions, metal salts or compounds containing metal-ions having antimicrobial properties may be applied to surfaces to impart an antimicrobial property to the surface. If, or when, the surface is inoculated with harmful microbes, the antimicrobial metal-ions or metal complexes, if present in effective concentrations, will slow or even prevent altogether the growth of those microbes. Antimicrobial activity is not limited to noble metals but is also observed in organic materials such as chlorophenol compounds (Triclosan™), isothiazolone (Kathon™), antibiotics, and some polymeric materials.

It has also been recognized that small concentrations of metal-ions may play an important role in biological processes. For example, Mn, Fe, Ca, Zn, Cu and Al are essential bio-metals, and are required for most, if not all, living systems. Metal-ions play a crucial role in oxygen transport in living systems, and regulate the function of genes and replication in many cellular systems. Calcium is an important structural element in the formation of bones and other hard tissues. Mn, Cu and Fe are involved in metabolism and enzymatic processes. At high concentrations, metals may become toxic to living systems and the organism may experience disease or illness if the level cannot be controlled. As a result, the availability and concentrations of metal-ions in aqueous and biological environments is a major factor in determining the abundance, growth-rate and health of plant, animal and microorganism populations.

It has been recognized that iron is an essential biological element, and that all living organisms require iron for survival and replication. Although the occurrence and concentration of iron is relatively high on the earth's surface, the availability of “free” iron is severely limited by the extreme insolubility of iron in aqueous environments. As a result, many organisms have developed complex methods of procuring “free” iron for survival and replication; and depend directly upon these mechanisms for their survival. The removal of iron from a system may inhibit the growth of such organisms.

Metal-ions may also exist as contaminants in environments such as drinking water, beverages, food, industrial effluents and public waste waters, and radioactive waste. Methods and materials for removing such contaminants are important for cleaning the environment(s) and providing for the safety of the general public. Materials such as filters, ion-exchange membranes, osmotic cleaners, etc. have been used to remove metal-ion contaminants, however, bio-fouling of these articles can be a major problem. Bio-fouling results from microbial growth in upon or near such articles, and may severely limit their effectiveness.

The use of a Fe chelatant (EDTA) in combination with an antimicrobial compound was described in Chidambaram, M.; Lok, Charmaine, Nephrology Rounds, (2003), Vol. 4, Issue 6. Further, it is generally known that multiplying stress factors will dramatically impact micro-organism growth (see for instance—General Stress Response of Bacillus subtilis and Other Bacteria.—Hecker, Michael; Volker, Uwe.—Advances in Microbial Physiology (2001), 44, 35-91).

U.S. Pat. No. 5,217,998 to Hedlund et al. describes a method for scavenging free iron or aluminum in fluids such as physiological fluids by providing in such fluids a soluble polymer substrate having a chelator immobilized thereon. A composition is described which comprises a water-soluble conjugate comprising a pharmaceutically acceptable water-soluble polysaccharide covalently bonded to deferoxamine, a known iron chelator. The conjugate is said to be capable of reducing iron concentrations in body fluids in vivo. The iron chelator is covalently bound to a soluble polymer and thus may not be easily or readily immobilized upon a substrate.

U.S. Pat. No. 6,156,234 to Meyer-Ingold et al. describes novel wound coverings which can remove interfering factors (such as iron ions) from the wound fluid of chronic wounds. The wound coverings may comprise iron chelators covalently bonded to a substrate such as cloth or cotton bandages.

U.S. Pat. No. 5,560,929 to Hedstrand et al. describes dense star polymers or dendrimers having a highly branched interior structure and capable of associating or chelating with metal-ions. Affinity for metal ions is achieved by modifying the dense star polymers with a plurality of oxygen and nitrogen atoms.

U.S. Pat. No. 5,854,303 to Powell et al. describes a polymeric material incorporating a polyvalent cation chelating agent in an amount effective to inhibit the growth of an ocular pathogen. The polymer of the invention may consist of a plurality of monomers, which are covalently modified with an agent capable of chelating a metal-ion, such as an alpha amino carboxylate.

U.S. Patent application U.S. 2003/0078209 A1 to Schmidt et al. describes solid porous compositions, substantially insoluble in water, comprising at least 25% by weight of an oxidized cellulose and having a significant capacity to bind iron. The invention also provides a method of sequestering dissolved iron from aqueous environments. The compositions may be used for the prevention or treatment of infections by bacteria or yeast.

U.S. Pat. No. 6,489,499 B1 to King et al. describes novel compounds comprising siloxane modified carboxylic acid substituted amines. The compounds of the invention are said to provide many of the desirable properties of ethylene diammine tetraacetic acid and its salts in a stationary phase. The stationary substrate may comprise silicate glass, silica, alumina, inorganic clays, etc. Markowitz et al. (J. Phys. Chem. B. 104, 10820 (2000)) describes metal chelating agents, such as carboxylic acid substituted amines, covalently bound to mesoporous silica. Tien (Chem. Mater. 11, 2141(1999)) describes silica gels functionalized with carboxylic acid substituted amines, the functionalization of which was accomplished using siloxane modified carboxylic acid substituted amines. The usefulness of the material “as a stationary phase for chromatographic separation of metal-ions” was demonstrated.

Tarasov and O'Hare (Inorganic Chemistry, 42, 1919 (2003)) have shown that alpha amino carboxylates such as ethylenediamminetetraacetate (EDTA) may be intercalated into layered double hydoxide to form intercalation complexes. The authors further show that soluble metals such as copper and nickel may be trapped into the solid phase of the intercalation complex. There is a problem, however, in that the alpha amino carboxylate intercalated does not have a sufficiently high affinity for metal-ions, and is not highly-selective for biologically important metal-ions.

U.S. Patent Application 0091767 A1 to Podhajny describes a method of applying an antimicrobial treatment to a packaging material, and to polymer dispersions containing antimicrobial zeolites. The zeolite containing dispersions may be formulated in water-based or solvent-based systems. Suitable polymers for practice of the invention listed are polyamides, acrylics, polyvinyl chloride, polymethyl methacrylates, polyurethane, ethyl cellulose, and nitro celluloses.

U.S. Pat. No. 5,556,699 to Niira et al describes transparent polymeric films containing antimicrobial zeolites which are ion exchanged with silver and other ions. The films are said to display antimicrobial properties. Polymeric materials suitable for the invention include ethylene ethyl acrylate (EEA), ethylene vinyl acetate (EVA), polyethylene, polyvinyl chlorides, polyvinyl fluoride resins, and others.

Materials are needed that can remove harmful contaminants from the environment and that can safely control the growth of microorganisms. Food and consumer packaging materials are needed that are able to improve food quality, to increase shelf-life, and to protect the contents from microbial contamination. Materials are also needed that are able to target and remove specific metal-ions from the surrounding environment, while leaving intact the concentrations of beneficial metal-ions. Furthermore, materials are needed that have a high capacity for metal-ions and which provide for the efficient removal of metal-ions in a cost effective manner. Materials and articles are needed that are able to effectively remove metal-ion contaminants, and are able to prevent bio-fouling of filtration devices.

### SUMMARY OF THE INVENTION

This invention provides a composition of matter comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion. It further provides an article comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion. It also provides a method of removing target metal-ions from an environment and releasing antimicrobial metal ions into the environment comprising contacting the environment with said composition.

This invention provides compositions that are able to sequester iron and other target metals and release metal ions which then act as an antimicrobial. The composition of the invention is able to target and remove specific metal-ions, while leaving intact the concentrations of beneficial metal-ions. The composition can be utilized to remove metal ions which are themselves contaminants, or they can be used to remove metal ions which are nutrients for biological contaminants. Thus, the composition provides two different methods of controlling the growth of biological contaminants. The composition has a high capacity for metal-ions and can provide for the efficient removal of metal-ions in a cost effective manner. The composition can also be used to effectively remove metal-ion contaminants without bio-fouling of the filtration devices

### DETAILED DESCRIPTION OF THE INVENTION

The composition of matter of the invention is useful for removing or sequestering target metal-ions from an environment. In many instances, it is necessary to remove metal-ions from environments such as drinking water, food, biological fluids, industrial effluents and public waste water, and radioactive waste. The composition of matter of the present invention may be applied to articles such as filters, sponges, membranes, textiles, fibres, plastics, metals, paper and other materials used in the construction of articles. Articles containing the composition of matter of the invention are placed in contact with the environment in an amount sufficient to bind the target metal-ion(s), and are then removed or separated from the environment, leaving the environment substantially free of the target metal-ion(s). Additionally, the composition of matter of the invention may prevent bio-fouling of materials, articles or devices used to remove metal-ion contaminants.

In a particular application of the invention, the composition of matter may be applied to the surfaces of consumer items such as plastic wraps, papers, cellophane and polymer films, glass and metal containers and other packaging materials, especially food packaging materials. The composition of matter of the invention may also be applied to medical items such as bandages, gauze, cotton and personal hygiene items such as diapers, band-aids, and other items which come into contact with biological and body fluids. In a particular embodiment, the composition of matter of the invention may be applied to such items such that it is transparent and un-noticeable to the user of the article. The composition of matter of the invention, and articles comprising the composition of matter of the invention are able to remove or sequester metal-ions such as Zn, Cu and Fe which are essential for biological growth, and thus may inhibit the growth of harmful micro-organisms such as bacteria, viruses, and fungi in the environment they contact. The invention “starves” the micro-organisms of minute quantities of essential nutrients and hence limits their growth and reduces the risk due to algal, fungal, bacterial, and other infectious diseases. In addition, It releases an antimicrobial metal ion which further acts to reduce the growth of microorganisms, and also extend the application of the invention to viruses, which are not impacted by nutrient starvation This concerted double-effect of the invention is especally important in environments which contain a high concentration, or dense population, of micro-organisms since the micro-organisms when healthy may bind strongly to bio-essential elements such as iron. The initial biocidal effect due to antimicrobial release helps to reduce the population of micro-organisms which, in turn, facilitates the sequestration process.

The invention provides a composition of matter comprising an immobilized metal-ion sequestrant/antimicrobial wherein said metal-ion sequestrant has a high stability constant for a target metal-ion and has attached thereto an antimicrobial metal-ion, wherein the stability constant of the metal-ion sequestrant for the antimicrobial metal-ion is less than the stability constant of the metal-ion sequestrant for the target metal-ion. The composition of matter contains an immobilized metal-ion sequestrant/antimicrobial. The term immobilized, as used herein, defines the metal-ion sequestrant/antimicrobial as being attached to a support, and as such, the metal-ion sequestrant/antimicrobial is not free to diffuse away from the support or to dissolve into the liquid medium in which the support is immersed. The metal-ion sequestrant/antimicrobial may be immobilized by means of a covalent chemical bond, or may be electrostatically immobilized on a support such as by mordant polymers, or may be immobilized via intercalation chemistry. The support may be, but is not limited to, a polymer, glass, paper, plastic, cellulose, textiles, metal or wood. It is preferred that the composition of matter is immobilized directly in or onto a support.

Preferred immobilized metal-ion sequestrants for practice of the invention have been described in detail in co-pending, commonly assigned, concurrently filed, U.S. patent application Ser. No. ______ (Docket 88079) and in U.S. patent application Ser. Nos. 10/822,940, 10/822,929 and 10/822,939 incorporated herein by reference. In one embodiment the immobilized metal-ion sequestrants include derivatized nanoparticles comprising inorganic nanoparticles having an attached metal-ion sequestraint, wherein said inorganic nanoparticles have an average particle size of less than 200 nm and the derivatized nanoparticles have a stability constant greater than 10¹⁰ with iron (III), described in U.S. patent application Ser. No. 10/822,940. It is preferred that the nanoparticles have an average particle size of less than 100 nm. It is further preferred that the nanoparticles have an average size of less than 50 nm, and most preferably less than 20 nm.

In another embodiment the immobilized metal-ion sequestrants are intercalated composite particles comprising a layered host material intercalated with a metal ion sequestrant having a stability constant greater than 10¹⁵ with iron (III), described in U.S. patent application Ser. No. 10/822,939. It is preferred that the layered host is chosen from layered double hydroxides. It is preferred that the stability constant of the sequestrant for iron(III) be greater than 10²⁰, and more preferably that that the metal-ion sequestrant has a stability constant for iron greater than 10³⁰.

In another embodiment the immobilized metal-ion sequestrants include a functionalized mordant polymer comprising a cationic polymer having an adsorbed metal-ion sequestrant, wherein the metal-ion sequestrant has a stability constant greater than 1010 with iron (III), described in co-pending, commonly assigned, concurrently filed U.S. Ser. No. ______ (Docket 88079) co-filed herewith. It is preferred that said cationic polymer comprises a quaternary ammonium or a quaternary phosphonium group. It is further preferred that said cationic polymer comprises a ploymer having the following composition: 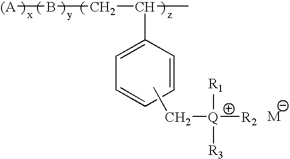
wherein: A represents units of an addition polymerizable monomer containing at least two ethylenically unsaturated groups; B represents units of a copolymerizable, α,β-ethylenically unsaturated monomer; Q is N or P; R₁, R₂ and R₃ each independently represents a carbocyclic or alkyl group; M⁻ is an anion; x is from 0.25 to 10 mole percent; y is from 0 to 90 mole percent; and z is from 10 to 99 mole percent. It is further preferred that said cationic polymer is a cationic latex mordant particle.

In one embodiment the composition of matter is immobilized on a particle. It is still further preferred that the thus formed metal-ion sequestrant/antimicrobial particle is immobilized on or in a support, such as a polymer, paper, metal, plastic, glass, wood, or textiles. It is still yet further preferred that the metal-ion sequestrant/antimicrobial particle is immobilized in a polymer layer.

When the metal-ion sequestrant/antimicrobial or metal-ion sequestrant/antimicrobial particles are immobilized in a polymer or polymer layer, in one embodiment the polymer comprises one or more of polyvinyl alcohol, polyethylene glycol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile or copolymers thereof.

The composition of matter of the invention comprises a metal-ion sequestrant having a high stability constant for a target metal-ion. Preferably said metal-ion sequestrant has a high-affinity for iron, copper, zinc, aluminum or heavy metals. The term heavy metals refers to metals having an atomic weight greater than about 100 g/mol, such as Ag, Au, Tl, Pb, Cd, and also lanthanides such as La, Ce, Sm, Eu, and Gd, and radioactive metals such as Th, U and Pu. It is also preferred that the metal-ion sequestrant has a high-affinity for biologically significant metal-ions, such as, Zn, Cu, Mn and Fe.

A measure of the “affinity” of metal-ion sequestrants for various metal-ions is given by the stability constant (also often referred to as critical stability constants, complex formation constants, equilibrium constants, or formation constants) of that sequestrant for a given metal-ion. Stability constants are discussed at length in “Critical Stability Constants”, A. E. Martell and R. M. Smith, Vols. 1-4, Plenum, N.Y. (1977), “Inorganic Chemetal-ion sequestranttry in Biology and Medicine”, Chapter 17, ACS Symposium Series, Washington, D.C. (1980), and by R. D. Hancock and A. E. Martell, Chem. Rev. vol. 89, p. 1875-1914 (1989). The ability of a specific molecule or ligand to sequester a metal-ion may depend also upon the pH, the concentrations of interfering ions, and the rate of complex formation (kinetics). Generally, however, the greater the stability constant, the greater the binding affinity for that particular metal-ion. Often the stability constants are expressed as the natural logarithm of the stability constant. Herein the stability constant for the reaction of a metal-ion (M) and a sequestrant or ligand (L) is defined as follows:
M+n L⇄MLₙ

where the stability constant is βₙ=[MLₙ]/[M][L]ⁿ, wherein [MLₙ] is the concentration of “complexed” metal-ion, [M] is the concentration of free (uncomplexed) metal-ion and [L] is the concentration of free ligand. The log of the stability constant is log βₙ, and n is the number of ligands which coordinate with the metal. It follows from the above equation that if βₙ is very large, the concentration of “free” metal-ion will be very low. Ligands with a high stability constant (or affinity) generally have a stability constant greater than 10¹⁰ or a log stability constant greater than 10 for the target metal. Preferably the ligands have a stability constant greater than 10¹⁵ for the target metal-ion. Table 1 lists common ligands (or sequestrants) and the natural logarithm of their stability constants (log βₙ) for selected metal-ions.

TABLE 1
Common ligands (or sequestrants) and the natural logarithm of their
stability constants (log βₙ) for selected metal-ions.

| Ligand | Ca | Mg | Cu(II) | Fe(III) | Al | Ag | Zn |
|---|---|---|---|---|---|---|---|
| alpha-amino | | | | | | | |
| carboxylates | | | | | | | |
| EDTA | 10.6 | 8.8 | 18.7 | 25.1 | | 7.2 | 16.4 |
| DTPA | 10.8 | 9.3 | 21.4 | 28.0 | 18.7 | 8.1 | 15.1 |
| CDTA | 13.2 | | 21.9 | 30.0 | | | |
| NTA | | | | 24.3 | | | |
| DPTA | 6.7 | 5.3 | 17.2 | 20.1 | 18.7 | 5.3 | |
| PDTA | 7.3 | | 18.8 | | | | 15.2 |
| citric Acid | 3.50 | 3.37 | 5.9 | 11.5 | 7.98 | 9.9 | |
| salicylic acid | | | | 35.3 | | | |
| Hydroxamates | | | | | | | |
| Desferrioxamine B | | | | 30.6 | | | |
| acetohydroxamic | | | | 28 | | | |
| acid | | | | | | | |
| Catechols | | | | | | | |
| 1,8-dihydroxy | | | | 37 | | | |
| naphthalene | | | | | | | |
| 3,6 sulfonic acid | | | | | | | |
| MECAMS | | | | 44 | | | |
| 4-LICAMS | | | | 27.4 | | | |
| 3,4-LICAMS | 16.2 | | | 43 | | | |
| 8-hydroxyquinoline | | | | 36.9 | | | |
| disulfocatechol | 5.8 | 6.9 | 14.3 | 20.4 | 16.6 | | |

EDTA is ehtylenediamine tetraacetic acid and salts thereof,
DTPA is diethylenetriaminepentaacetic acid and salts thereof,
DPTA is Hydroxylpropylenediaminetetraacetic acid and salts thereof,
NTA is nitrilotriacetic acid and salts thereof,
CDTA is 1,2-cyclohexanediamine tetraacetic acid and salts thereof,
PDTA is propylenediammine tetraacetic acid and salts thereof.
Desferrioxamine B is a commercially available iron chelating drug, desferal ®.
MECAMS, 4-LICAMS and 3,4-LICAMS are described by Raymond et al. in “Inorganic Chemetal-ion sequestranttry in Biology and Medicine”, Chapter 18, ACS Symposium Series, Washington, D.C. (1980).
Log stability constants are from “Critical Stability Constants”, A. E. Martell and R. M. Smith, Vols. 1-4, Plenum Press, NY (1977); “Inorganic Chemetal-ion sequestranttry in Biology and Medicine”, Chapter 17, ACS Symposium Series, Washington, D.C. (1980); R. D. Hancock and A. E. Martell, Chem. Rev. vol. 89, p. 1875-1914 (1989) and “Stability Constants of Metal-ion Complexes”, The Chemical Society, London, 1964.

In some instances, it may be necessary to remove specific metal-ion(s) from a target environment. The target environment is a liquid environment, e.g., water, public waste water, industrial effluents, food or food extrudates, biological or physiological fluids. In such cases it may be desirable to immobilize a metal-ion sequestrant/antimicrobial with a very high specificity or selectivity for a given metal-ion. Immobilized metal-ion sequestrant/antimicrobials of this nature may be used to control the concentration of the target metal-ion and thus treat pollution, disease or illness associated with this metal-ion. One skilled in the art may prepare such immobilized metal-ion sequestrant/antimicrobials by selecting a metal-ion sequestrant having a high specificity for the target metal-ion. The specificity of a metal-ion sequestrant for a target metal-ion is given by the difference between the log of the stability constant for the target metal-ion, and the log of the stability constant for the interfering metal-ions. For example, if a treatment required the removal of Fe(III), but it was necessary to leave the Ca-concentration unaltered, then from Table 1, DTPA would be a suitable choice since the difference between the log stability constants 28-10.8=17.2, is very large. 3,4-LICAMS would be a still more suitable choice since the difference between the log stability constants 43-16.2=26.8, is the largest in Table 1.

It is preferred that the immobilized metal-ion sequestrants have a high stability constant for the target metal-ion(s). The stability constant for the immobilized metal-ion sequestrant/antimicrobials will largely be determined by the stability constant for the attached metal-ion sequestrant. However, The stability constant for the immobilized metal-ion sequestrant/antimicrobial s may vary somewhat from that of the attached metal-ion sequestrant. Generally, it is anticipated that metal-ion sequestrants with high stability constants will give immobilized metal-ion sequestrants/antimicrobial with high stability constants. For a particular application, it may be desirable to have an immobilized metal-ion sequestrant/antimicrobial with a high selectivity for a particular metal-ion. In most cases, the immobilized metal-ion sequestrant/antimicrobial will have a high selectivity for a particular metal-ion if the stability constant for that metal-ion is about 10⁶ greater than for other ions present in the system.

It is preferred that the immobilized metal-ion sequestrant/antimicrobial of the invention has a high-affinity for iron, and in particular iron(III). It is preferred that the stability constant of the sequestrant for iron(III) be greater than 10¹⁰. It is still further preferred that the metal-ion sequestrant has a stability constant for iron greater than 10²⁰. It is still further preferred that the metal-ion sequestrant has a stability constant for iron greater than 10³⁰.

Metal-ion sequestrants may be chosen from various organic molecules. Such molecules having the ability to form complexes with metal-ions are often referred to as “chelators”, “complexing agents”, and “ligands”. Certain types of organic functional groups are known to be strong “chelators” or sequestrants of metal-ions. It is preferred that the sequestrants of the invention contain alpha-amino carboxylates, hydroxamates, or catechol, functional groups. Hydroxamates, or catechol, functional groups are preferred. Alpha-amino carboxylates have the general formula:
R—[N(CH₂CO₂M)—(CH₂)ₙ—N(CH₂CO₂M)₂]ₓ
where R is an organic group such as an alkyl or aryl group; M is H, or an alkali or alkaline earth metal such as Na, K, Ca or Mg, or Zn; n is an integer from 1 to 6; and x is an integer from 1 to 3. Examples of metal-ion sequestrants containing alpha-amino carboxylate functional groups include ethylenediaminetetraacetic acid (EDTA), ethylenediaminetetraacetic acid disodium salt, diethylenetriaminepentaacetic acid (DTPA), Hydroxylpropylenediaminetetraacetic acid (DPTA), nitrilotriacetic acid, triethylenetetraaminehexaacetic acid, N,N′-bis(o-hydroxybenzyl) ethylenediamine-N,N′diacteic acid, and ethylenebis-N,N′-(2-o-hydroxyphenyl)glycine.

Hydroxamates (or often called hydroxamic acids) have the general formula: 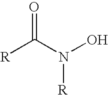
where R is an organic group such as an alkyl or aryl group. Examples of metal-ion sequestrants containing hydroxamate functional groups include acetohydroxamic acid, and desferroxamine B, the iron chelating drug desferal.

Catechols have the general formula: 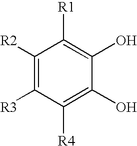
where R1, R2, R3 and R4 may be H, an organic group such as an alkyl or aryl group, or a carboxylate or sulfonate group. Examples of metal-ion sequestrants containing catechol functional groups include catechol, disulfocatechol, dimethyl-2,3-dihydroxybenzamide, mesitylene catecholamide (MECAM) and derivatives thereof, 1,8-dihydroxynaphthalene-3,6-sulfonic acid, and 2,3-dihydroxynaphthalene-6-sulfonic acid.

The immobilized metal-ion sequestrant/antimicrobial of the invention has an attached antimicrobial metal-ion. The primary mode of attachment of the attached antimicrobial metal-ion is via chelation (or complexation) of the antimicrobial metal-ion by the immobilized metal-ion sequestrant. The antimicrobial metal-ion may also be “attached” or contained within the inventive composition by traditional means such as via an ion-exchange reagent, e.g., zeolites, metal hydrogen phosphate, or clay, or by other means. The antimicrobial metal-ion might also be contained within a polymer, or a polymeric layer, or imbided into cellulose. The antimicrobial metal-ion may be in its ionic form, M⁺, or in its metallic form, M°, which may then dissolve into the liquid medium. These and other methods are discussed in co-pending, commonly assigned, concurrently filed, U.S. patent application Ser. No. ______ (Docket # 86559A) co-filed herewith. The stability constant of the metal-ion sequestrant for the antimicrobial metal-ion is lower than that for the target metal-ion. Therefore, upon exposure to a liquid medium containing the target metal-ion(s), the immobilized metal-ion sequestrant will exchange the antimicrobial metal-ion for the target metal-ion according to eqn 1.
IMIS(AM)+TM⇄IMIS(TM)+AM (1)
where IMIS is the immobilized metal-ion sequestrant, AM is the antimicrobial metal-ion and TM is the target metal-ion. Because the stability constant of the immobilized metal-ion sequestrant is greater for the target metal-ion, the equilibrium will lie to the right of eqn. 1. The antimicrobial metal-ion may also be exchanged for metal-ions other than the target metal-ion, depending upon the concentration and stability constant of the sequestrant for the other metal-ion. The antimicrobail metal-ion will therefore be free to diffuse into the liquid medium and may inhibit the growth of, or kill, micro-organisms contained therein. It is preferred that the antimicrobial metal-ion is selected from one or more of silver, copper, nickel, zinc, gold and tin. It is further preferred that the antimicrobial metal-ion is silver.

This invention also comprises an article comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion. The immobilized metal-ion sequestrant/antimicrobial is as described above.

The immobilized metal-ion sequestrant/antimicrobial may be contained in a polymer layer, said layer being located on the surface of the article. It may also be incorporated into the materials forming the article. The metal-ion sequestrant/antimicrobial may be immobilized on particles which are then incorporated into the materials forming the article or are immobilized in a polymer layer, said layer being located on the surface of the article. The article may be comprised of, for example, paper, metal, plastic, glass, wood, or textiles.

When a polymer layer is utilized it may comprise one or more of polyvinyl alcohol, polyethylene glycol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile or copolymers thereof.

The invention may also comprise a barrier layer; wherein the polymeric layer is between the surface of the article and the barrier layer and wherein the barrier layer does not contain the immobilized metal-ion sequestrant/antimicrobial. The barrier layer may provide several functions including improving the physical strength and toughness of the article and resistance to scratching, marring, cracking, etc. However, the primary purpose of the barrier layer is to provide a barrier through which micro-organisms cannot pass. It is important to limit, or eliminate, the direct contact of micro-organisms with the surfaces of the immobilized metal-ion sequestrant/antimicrobial, since many micro-organisms, under conditions of iron deficiency, may bio-synthesize molecules which are strong chelators for iron, and other metals. These bio-synthetic molecules are called “siderophores” and their primary purpose it to procure iron for the micro-organisms. Thus, if the microorganism are allowed to directly contact the immobilized metal-ion sequestrant/antimicrobial of the invention, they may find a rich source of iron there, and begin to colonize directly at these surfaces. The siderophores produced by the micro-organisms may compete with the immobilized metal-ion sequestrant/antimicrobial for the iron (or other bio-essential metal) at their surfaces. The barrier layer of the invention does not contain an immobilized metal-ion sequestrant/antimicrobial, and because micro-organisms are large, they may not pass or diffuse through the barrier layer. The barrier layer thus prevents contact of the micro-organisms with the polymeric layer containing the immobilized metal-ion sequestrant/antimicrobial of the invention.

It is preferred that the barrier layer is permeable to liquid media. This is preferred because metal-ions in solution may then readily diffuse through the barrier layer and become sequestered in the underlying polymeric layer containing the derivatized nanoparticles. Thus, the barrier layer spatially separates the micro-organisms from the polymeric sequestration layer. It is preferred that the polymer(s) of the barrier layer has a water permeability of greater than 1000 [(cm³ cm)/(cm²sec/Pa)]×10¹³. It is further preferred that the polymer(s) of the barrier layer has a water permeability of greater than 5000 [(cm³ cm)/(cm²sec/Pa)]×10¹³. Preferred polymers for use in the barrier layer are one or more of polyvinyl alcohol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene, polypropylene, or polyacrylonitrile or copolymers thereof. It is preferred that the barrier layer has a thickness in the range of 0.1 microns to 10.0 microns. Barrier layers are discussed at length in U.S. patent application Ser. No. 10/822,929.

The invention further provides a method of removing target metal-ions from an environment and releasing antimicrobial metal ions into the environment comprising contacting the environment with a composition comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion. In a preferred embodiment the environment is a liquid medium. Preferably the target metal-ion concentration in the liquid medium is reduced to less than 100 ppb. In one embodiment the target metal ion is iron. Preferably the iron concentration in the liquid medium is reduced to less than 50 ppb.

The following examples are intended to illustrate, but not to limit, the invention.

### EXAMPLES

Preparation of Derivatized Nanoparticles:

Colloidal dispersions of silica particles were obtained from ONDEO Nalco Chemical Company. NALCO® 1130 had a median particle size of 8 nm, a pH of 10.0, a specific gravity of 1.21 g/ml, a surface area of about 375 m²/g, and a solids content of 30 weight. N-(trimethoxysilylpropylethylenediamine triacetic acid, trisodium salt was purchased from Gelest Inc., 45% by weight in water.

Preparation of derivatized nanoparticles. To 600.00 g of silica NALCO® 1130 (30% solids) was added 400.00 g of distilled water and the contents mixed thoroughly using a mechanical mixer. To this suspension, was added 49.4 g of N-(trimethoxysilyl)propylethylenediamine triacetic acid, trisodium salt in 49.4 g distilled water with constant stirring at a rate of 5.00 ml/min. At the end of the addition the pH was adjusted to 7.1 with the slow addition of 13.8 g of concentrated nitric acid, and the contents stirred for an hour at room temperature. Particle size analysis indicated an average particle size of 15 nm. The percent solids of the final dispersion was 18.0%.

Preparation of the immobilized metal-ion sequestrant/antimicrobial: 200.0 g of the above derivatized nanoparticles were washed with distilled water via dialysis using a 6-8000 molecular weight cutoff filter. The final ionic strength of the solution was less than 0.1 millisemens. To the washed suspension was then added with strirring 4.54 ml of 1.5 M AgNO₃ solution, to form the immobilized metal-ion sequestrant/antimicrobial.

Preparation of Polymeric Layers of Immobilized Metal-Ion Sequestrant/Antimicrobial.

Coating 1. A coating solution was prepared as follows: 12.8 grams of the immobilized metal-ion sequestrant/antimicrobial suspension prepared as described above was combined with to 77.4 grams of pure distilled water and 8.8 g of a 40% solution of the polyurethane Permax 220 (Noveon Chemicals). 1.0 g of a 10% solution of the surfactant OLIN 10 G was added as a coating aid. The mixture was then stirred and blade-coated onto a polymeric support using a 150 micron doctor blade. The coating was then dried at 40-50° C., to produce a film having 2.7 g/m² of the immobilized metal-ion sequestrant/antimicrobial, 0.06 g/m² silver-ion and 5.4 g/m² of polyurethane.

Coating 2. A coating solution was prepared as follows: 8.8 g of a 40% solution of the polyurethane Permax 220 (Noveon Chemicals) was combined with to 90.2 grams of pure distilled water and 1.0 g of a 10% solution of the surfactant OLIN 10G was added as a coating aid. The mixture was then stirred and blade-coated onto a polymeric support using a 150 micron doctor blade. The coating was then dried at 40-50° C., to produce a film having 5.4 g/m² of polyurethane.

### Example 1 and Comparison Example 1

Testing Methodology

Since the efficacy of the antimicrobial agent is determined by the mechanism of action, which is determined by the kind of Micro-organism and environment of use, it is critical that the test procedure that is used is well defined and consistent with the procedures used in the literature.

Testing of antimicrobial activity is based on the contact between the antimicrobial substance and the targeted microorganisms followed by the measurement of the impact on the microorganism viability or growth rate. In the case of surfaces, surface treatment or coatings integrating antimicrobials, the activity relies on the diffusion of the antimicrobial from the surface to the environment in which micro-organisms are present The selection of micro-organisms that are tested is based on the mechanisms of Ag activity that are well described in literature (see for instance—The molecular mechanisms of copper and silver ion disinfection of bacteria and viruses.—R. B. Thurman & C. P. Gerba—CRC Critical Reviews in Environmental Control, vol. 18(4), p295-315, 1989.). The key parameter of Ag activity is molecular binding to the cell wall that usually stops micro-organism growth, without killing it. Then, Ag diffusion inside the cell usually induces cell death by Ag complexing with enzymes and even nucleic acids. All these mechanisms require interaction with the cell wall and the cell membrane. Therefore, sensitivity of micro-organisms to Ag antimicrobial activity mainly relies on cell wall types. It is well known that gram negative bacteria, having a more complex cell wall structure than gram positive, are less sensitive to antimicrobials and especially Ag. This is also the case of molds that have a cellulosic cell wall that is more resistant to Ag activity, Matsumura et al. list data showing different activities of Ag for various micro-organsims. (Mode of Bactericidal Action of Silver Aeolite and its Comparison with that of Silver Nitrate.—Matsumura, Yoshinobu; Yoshikata, Kuniaki; Kunisaki, Shin-ichi; Tsuchido, Tetsuaki.—Applied and Environmental Microbiology (2003), 69(7), 4278-4281).

Focusing on antimicrobials, so applied to micro-organsims, the following main categories of organisms were selected according to their cell wall characteristics:
viruses: either nacked or encapsulated and RNA or DNA
bacteria: gram negative, gram positive, mychobacteria
fungi: molds and yeast

For the specific testing of metal starvation combined to heavy metal antimicrobial activity a yeast, a bacteria and a mold were selected as the main micro-organisms, because of their different cell wall structures.

Specific operating conditions were selected in the range of conditions for heterotrophic aerobic growth Trypcase Soy Agar or Sabouraud Dextrose Agar was used. The appropriate temperature for the microorganism or for the application was selected for instance at 30° C. The yeast used was Candida albicans (ATCC-10231) which is a common yeast involved in various human deseases. The bacteria was Escherichia coli (ATCC-11775), a typical bacteria used in antimicrobial testing and the mold was Paecilomyces variotii (UMIP 1024.7) a typical fast growing mold often isolated in beverages.

Viruses were not tested as they will not be impacted by Fe starvation, but only by Ag release, which is already described in the literature.

The quantitative method for antimicrobial diffusing from a surface was the dipping test. Operating conditions can be very different according to the targeted application. Some are normalized in standard methods, for instance: AATCC-100 for the quantitative evaluation of textiles treated with antimicrobial finishes, JIS Z-2801 for the test of activity and efficacy of antimicrobial products, ASTM-E2149-01 for determining the antimicrobial activity of immobilized antimicrobial agents under dynamic contact conditions, and ASTM E 2180-01 for determining the activity of incorporated antimicrobial agent(s) in polymeric or hydrophobic materials. The principle of these methods is to put a piece of surface (coating, textiles, etc.) with a known surface area in contact with a solution inoculated with microorganisms. The leaching of antimicrobials from the surface into the solution impacts the microorganism growth when the antimicrobial activity is significant. Following microorganism number in solution (planktonic) over time allows quantitative evaluation of the antimicrobial activity.

For long contact time, more than 1 day, operating conditions were selected in order to best fit a range of potential applications. A piece of coating with 1×1 cm area was used. The coating was dipped in 2 ml of liquid growth medium, inoculated with a final concentration of 2 000 yeasts/ml, 13 000 bacteria/ml or 1 000 mold spore/ml. The growth medium was Trypcase Soy broth diluted 1/10 in sterile water for yeast and bacteria. It was Sabouraud broth diluted 1/10 for molds.

Special attention was made to all reagents to avoid accidental Fe contamination. Especially the dilution water was so called ultrapure, with lower than 1 ppb of Fe. The Micro-organism number in the solution was measured daily over several days, or at regular time intervals over 1 day, by the standard heterotrophic plate count method, respectively on Trypcase Soy Agar (TSA). Results are presented in Colony Forming Units/ml (or CFU/ml) versus time for a given incubation temperature, a given coating and a given microorganism. The lower the number of micro-organsim, the higher the antimicrobial activity (see Table 2).

TABLE 2

| | | Yeast count | Yeast count | Yeast count |
|---|---|---|---|---|
| | | at time (0 | at time (24 | at time (48 |
| | | hours) | hours) | hours) |
| Examples | Material | (CFU/ml) | (CFU/ml) | (CFU/ml) |
| coating | immobilized | 2,000 | not | not |
| sample 1 | sequestrant/ | | detectable | detectable |
| | antimicrobial | | | |
| coating | No immobilized | 2,000 | 2,000,000 | 5,000,000 |
| sample 2 | sequestrant/ | | | |
| | antimicrobial | | | |

The results of the examples show that the the coatings containing the immobilized sequestrant/antimicrobial have a fungocidal effect upon the yeast populations, and reduce the yeast concentration in the growth medium to an undetectable level. The coating sample which does not contain the immobilized sequestrant/antimicrobial does not inhibit the growth of the yeast populations. The same experiment was performed on the bacteria (E. coli) and the mold (P. variotii) selected. They were both tested using same operationg conditions as listed above. Results are presented in Table 3 and 4.

TABLE 3

| | | Bacteria | | |
|---|---|---|---|---|
| | | count at | | |
| | | time (0 | Bacteria | Bacteria |
| | | hours) | count at time | count at time |
| | | (CFU/ | (24 hours) | (48 hours) |
| Examples | Material | ml) | (CFU/ml) | (CFU/ml) |
| coating | immobilized | 13,000 | not | not |
| sample 1 | sequestrant/ | | detectable | detectable |
| | antimicrobial | | | |
| coating | No immobilized | 13,000 | >100,000,000 | >100,000,000 |
| sample 2 | sequestrant/ | | | |
| | antimicrobial | | | |

The results of the examples show that the the coatings containing the immobilized sequestrant/antimicrobial have a bacteriocidal effect upon the bacteria population, and reduce the bacteria concentration in the growth medium to an undetectable level. The coating sample which does not contain the immobilized sequestrant/antimicrobial does not inhibit the growth of the bacteria population.

For mold cultivation, only a visual observation, by one skilled in the art of microbiology, was used. Results are expressed as “Not detectable” for negative tubes without a biogrowth, or “Growth” for positive tubes with significant biogrowth.

TABLE 4

| | | Fungi | Fungi | |
|---|---|---|---|---|
| | | growth at | growth at | Fungi growth |
| | | time (0 | time (48 | at time (96 |
| Examples | Material | hours) | hours) | hours) |
| coating | immobilized | not | not | not |
| sample 1 | sequestrant/ | detectable | detectable | detectable |
| | antimicrobial | | | |
| coating | No immobilized | not | Growth | Growth |
| sample 2 | sequestrant/ | detectable | | |
| | antimicrobial | | | |

The results of the examples show that the the coatings containing the immobilized sequestrant/antimicrobial have a fungocidal effect upon the mold population, and reduce the mold concentration in the growth medium to an undetectable level. The coating sample which does not contain the immobilized sequestrant/antimicrobial does not inhibit the growth of the mold population.

The invention has been described in detail with particular reference to certain preferred embodiments thereof, but it will be understood that variations and modifications can be effected within the spirit and scope of the invention.

1. A composition of matter comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion.

2. The composition of matter of claim 1 wherein the metal-ion sequestrant has a stability constant greater than 10¹⁰ for iron (III).

3. The composition of matter of claim 2 wherein said metal-ion sequestrant has a stability constant with iron greater than 10²⁰.

4. The composition of matter of claim 2 wherein said metal-ion sequestrant has a stability constant with iron greater than 10³⁰.

5. The composition of matter of claim 1 wherein said metal-ion sequestrant comprises an alpha amino carboxylate, a hydroxamate, or a catechol functional group.

6. The composition of matter of claim 1 wherein said metal-ion sequestrant has a high stability constant with copper, zinc, aluminum or heavy metals.

7. The composition of claim 1 wherein said antimicrobial ions are metal ions selected from silver, copper, nickel, zinc, gold and tin.

8. The composition of claim 1 wherein said antimicrobial metal-ion is silver.

9. The composition of claim 5 wherein said antimicrobial metal-ion is silver.

10. The composition of claim 1 wherein the metal-ion sequestrant/antimicrobial is immobilized directly in or on a support.

11. The composition of claim 1 wherein the metal-ion sequestrant/antimicrobial is immobilized on particles.

12. The composition of claim 11 wherein the metal-ion sequestrant/antimicrobial particles are immobilized on or in a support.

13. The composition of claim 10 wherein the support is a polymer, paper, metal, plastic, glass, wood, or textiles.

14. The composition of claim 1 wherein said immobilized metal-ion sequestrant/antimicrobial is contained within a polymer

15. The composition of claim 14 wherein the polymer comprises one or more of polyvinyl alcohol, polyethylene glycol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile or copolymers thereof.

16. An article comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion.

17. The article of claim 16 wherein the immobilized metal-ion sequestrant/antimicrobial is contained in a polymer layer, said layer being located on the surface of the article.

18. The article of claim 16 wherein the immobilized metal-ion sequestrant/antimicrobial is incorporated into the materials forming the article.

19. The article of claim 16 wherein the metal-ion sequestrant/antimicrobial is immobilized on particles.

20. The article of claim 19 wherein the metal-ion sequestrant/antimicrobial particles are incorporated into the materials forming the article.

21. The article of claim 19 wherein the metal-ion sequestrant/antimicrobial particles are immobilized in a polymer layer, said layer being located on the surface of the article.

22. The article of claim 16 wherein the article is comprised of paper, metal, plastic, glass, wood, or textiles.

23. The article of claim 17 wherein the polymer layer comprises one or more of polyvinyl alcohol, polyethylene glycol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile or copolymers thereof.

24. The article of claim 21 wherein the polymer layer comprises one or more of polyvinyl alcohol, polyethylene glycol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile or copolymers thereof.

25. The article of claim 16 wherein said metal-ion sequestrant has a stability constant with iron greater than 10²⁰.

26. The article of claim 16 wherein said metal-ion sequestrant has a stability constant with iron greater than 10³⁰.

27. The article of claim 16 wherein said metal-ion sequestrant comprises an alpha amino carboxylate, a hydroxamate, or a catechol functional group.

28. The article of claim 16 wherein said metal-ion sequestrant has a high stability constant with copper, zinc, aluminum or heavy metals.

29. The article of claim 16 wherein the antimicrobial ions are metal ions selected from silver, copper, nickel, zinc, gold and tin.

30. The article of claim 16 wherein said antimicrobial metal-ion is silver.

31. The article of claim 27 wherein said antimicrobial metal-ion is silver.

32. A method of removing target metal-ions from an environment and releasing antimicrobial metal ions into the environment comprising contacting the environment with a composition comprising an immobilized metal-ion sequestrant/antimicrobial comprising a metal-ion sequestrant that has a high stability constant for a target metal ion and that has attached thereto an antimicrobial metal-ion, wherein the stability constant of the metal-ion sequestrant for the antimicrobial metal-ion is less than the stability constant of the metal-ion sequestrant for the target metal-ion.

33. The method of claim 32 wherein the environment is a liquid medium.

34. The method of claim 33 wherein the target metal-ion concentration in the liquid medium is reduced to less than 100 ppb.

35. The method of claim 32 wherein the target metal ion is iron.

36. The method of claim 35 wherein the iron concentration in the liquid medium is reduced to less than 50 ppb.

37. The method of claim 32 wherein said metal-ion sequestrant has a stability constant for iron greater than 10²⁰

38. The method of claim 32 wherein said metal-ion sequestrant has a stability constant for iron greater than 10²⁰.

39. The method of claim 32 wherein said metal-ion sequestrant has a stability constant for iron greater than 10³⁰.

40. The method of claim 32 wherein said metal-ion sequestrant comprises an alpha amino carboxylate functional group.

41. The method of claim 32 wherein the antimicrobial ions are metal ions selected from silver, copper, nickel, zinc, gold and tin.

42. The method of claim 32 wherein said antimicrobial metal-ion is silver.
